(19) United States
(12) Patent Application Publication
(10) Pub. No.: US 2003/0097143 A1
(43) Pub. Date: May 22, 2003

(54) Multi-bladed surgical scalpel

(76) Inventor: Mark Mittelstaedt, Tucson, AZ (US)

Correspondence Address: MARK MITTELSTAEDT, 5941 E FORT CRITTENOON, TUCSON, AZ, 85750, US

(21) Appl. No.: 09/988,443

(22) Filed: Nov. 20, 2001

(51) Int. Cl. A61B 17/32

(52) U.S. Cl. 606/172

(57) ABSTRACT

This multi-bladed scalpel addresses the problem of making many small incisions in very close proximity (as close as 0.030″/0.75 mm) to each other, to facilitate hair transplantation. With this device, it is possible to make multiple incisions in such proximity. With the blades mounted parallel to each other, at the desired spacing, each incision does not intersect neighboring incisions, so the follicles placed in each incision will be surrounded by the maximum amount of undisturbed tissue to promote revascularization—capillary growth to provide a blood supply to each transplanted follicle. CNC machining techniques are used to create the blade holders with blade mounting sites created at the desired proximity. Medical grade epoxy is used to mount the blades, which are mounted parallel to each other. Holes are drilled so the tips of the blade group are located in a plane at an angle with respect to the blade holder for ergonomic reasons

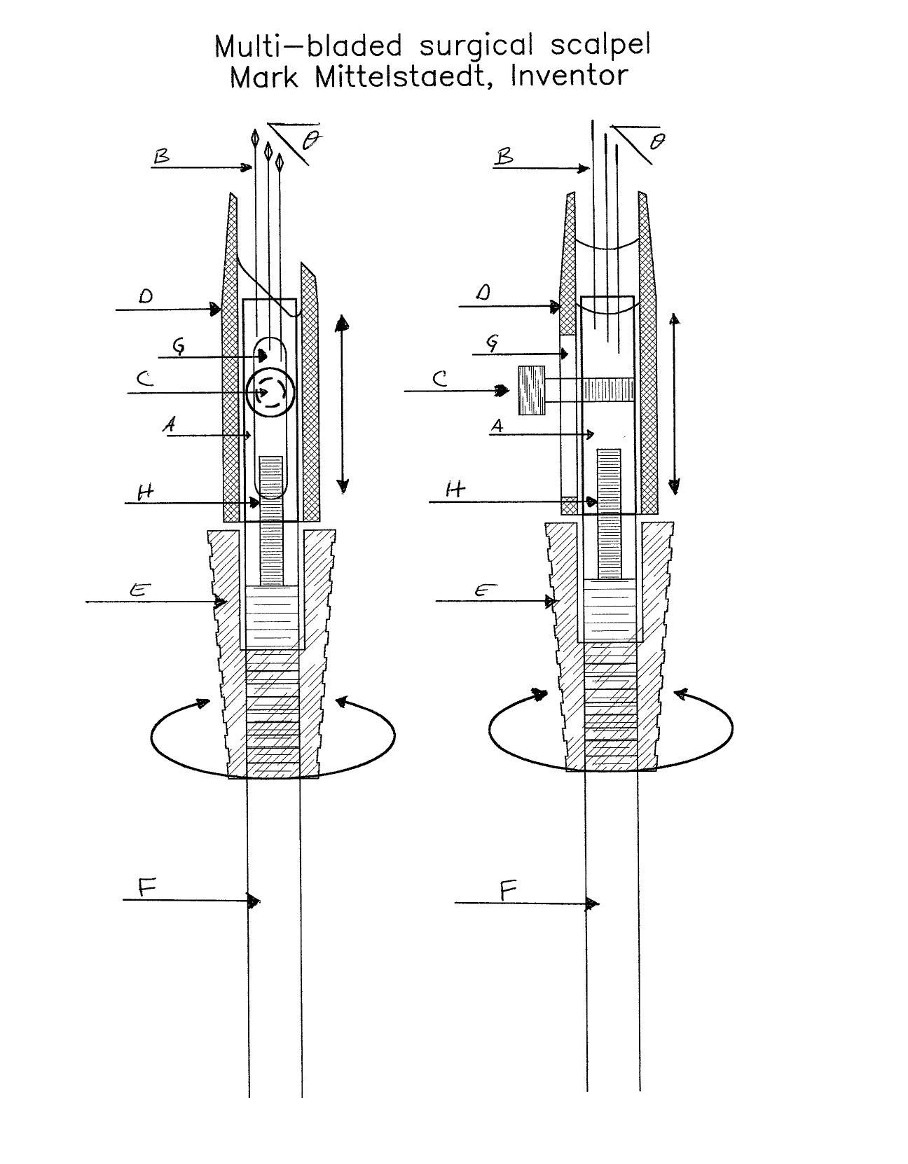

This multi-bladed surgical scalpel was created to facilitate hair transplantation. In this process, it is necessary to make a series of incisions in very close (as close as 0.030″ spacing between neighboring blades) proximity to each other, so hair follicles can be placed in each incision where they will then take root and grow hair.

In this embodiment, the blade holder is made from a section of Delrin rod, one inch long by 1/4″ in diameter. A series of holes just slightly (0.001″-0.003″) larger than the blade shaft diameters is drilled into the circular end face of the section. The holes are drilled such that the tips of all the blades are in the same plane, such plane to be at a desired angle to the end face of the section. A shallow (0.030″) orientation flat is machined on the side of the section for 0.10″ from the end face of the section into which the holes are drilled. The blades are mounted with medical grade epoxy, and oriented such that they are parallel to the orientation flat, with the blade edges facing the same direction and parallel to the orientation flat.

On the same side of the section as the orientation flat, a 4×40 hole is tapped midway along the length of the section, such that it is perpendicular to the plane of the orientation flat.

This Delrin section has a 4×40 hole tapped into the end face opposite the blades, into which is threaded a setscrew. This setscrew is then used to attach the section to a handle made of some other material, into which the same size setscrew hole has been threaded. There is an externally threaded section—in this case, ¼×20, immediately below the location onto which the Delrin section is mounted.

Prior to mounting the Delrin section, an adjustment nut is threaded onto the ¼×20 threads. It threads down so that the entire Delrin section is visible from the side. Then, the Delrin section is mounted. After that, a depth gauge is located over the Delrin section. This depth gauge is machined from tubing with an inside diameter 0.020″ larger than the Delrin section. It has a machined top angle corresponding to the angle of the plane of the blade tips. It is machined such that the wall thickness of the tubing at the top of the gauge is 0.030″ thick, and, grooves are machined in all four “sides” (left, right, front and back) at the top of the gauge, to allow better visibility of blade tips for blade group placement.

This gauge also has a slot machined into the side of the tubing, such that when the gauge slides over the Delrin section, the angle at the top of the gauge corresponds to the angle of the blade tips and the slot lines up with the 4×40 threaded hole in the side of the Delrin section. A 4×40 thumbscrew is now threaded through the slot in the side of the depth gauge, into the Delrin section, but not fully tightened. The depth gauge is adjusted by threading the adjustment nut up and down to expose the desired amount of blade to determine incision depth. When incision depth is set, the thumbscrew is tightened Assembly is now complete, and precise incisions can be made.

### CROSS REFERENCE TO RELATED APPLICATIONS

I found many tools to facilitate the process of making incisions for hair transplants, but none of them were simple, and none of them provided the desired blade spacing, angle, or orientation alignment. There are various mechanisms that contain motors and/or lasers. Typical of these are U.S. Pat. Nos. 5,817,120, 5,782,851, 5,584,841, 5,989,279, 6,022,345, 5,908,417, and 5,733,278, etc. There are simpler mechanisms, as well, notably U.S. Pat. No. 5,989,273. However, U.S. Pat. No. 5,989,273 is designed to create strips of scalp from which hair follicles to be transplanted are harvested. The blades in U.S. Pat. No. 5,989,273 are secured by a common mounting location and require spacers that provide a uniform distance between slices. It is not designed to make small, individual incisions. Both U.S. Pat. Nos. 5,026,385 and 5,447,516 are similar, and referenced by U.S. Pat. No. 5,989,273. They are designed to take uniform thin slices for the purpose of obtaining hair graft tissues.

THIS RESEARCH WAS NOT FUNDED IN ANY WAY BY THE FEDERAL GOVERNMENT. WE RETAIN ALL RIGHTS TO THIS INVENTION.

### BACKGROUND—FIELD OF INVENTION

This is a multi-bladed surgical scalpel that makes many tiny incisions in one motion. We could not find anything like it, so I devised this item that can be easily manufactured to provide many blades in a very small area, with blade spacing as close as 0.030″.

### OPERATION

The surgeon sets the desired depth of cut by adjusting the depth gauge, then places the scalpel where a series of small incisions is desired. Multiple incisions are then made with each stroke of the scalpel.

### INVENTION SUMMARY

Hair transplantation is often performed by a surgical procedure wherein some of a given patient's hair follicles are “harvested” from areas where the patient has remaining hair, then transplanted to bald or balding areas. In some procedures, the follicles to be transplanted are obtained from a donor “strip” of the patient's remaining hair. This strip is divided into follicular units, which are then placed in incisions. This multi-bladed scalpel provides randomly spaced incisions, at a spacing that simulates the appearance of natural scalp when follicles are transplanted.

With conventional single-bladed knives, a separate stroke is required to make each incision. Painstaking care must be taken to locate each incision at the desired location, make each incision to the desired depth, and align each incision such that it is made at the proper angle into the scalp. In surgery, this process is very time consuming. With multi-bladed scalpels, the amount of time required to make all the necessary incisions is greatly reduced. This reduces the amount of time a transplant patient has to spend under anesthesia.

In one embodiment of this design, an adjustable depth gauge is built into the handle of the knife. This allows the surgeon to set a uniform penetration depth, making it very easy to control the depth of each incision. Incisions that are too deep increase bleeding. They result in more post-surgical facial swelling, and take longer to heal. Graft compression may occur when an individual graft is too large for an incision. Grafts are often rejected from too-shallow incisions. Incisions that are too shallow are less effective at providing sufficient volume to insert follicular units.

Since incision depth and alignment are of such importance, a surgeon needs to be very painstaking with each incision. To provide a natural appearance, the incisions often need to be less than one millimeter/0.040″ apart. Such precise cuts quickly result in hand fatigue, and introduce the potential for repetitive stress injury to the surgeon. With the depth gauge, and with multiple cuts being made in one stroke, the percentage of cuts made at the ideal depth, spacing and alignment will greatly increase, thus increasing donor hair “yield”—the number of grafts that “take”, with minimum patient discomfort.

In various embodiments of this invention, different numbers of blades are used, and spacing between the blades is changed, to provide varying incision density. This allows the surgeon to more closely match the number of incisions with coverage area desired and amount of donor hair available.

Also, in various embodiments, the blade tips are mounted at varying angles. This allows the blade tips of a multi-bladed tool to make their individual incisions at the same time when the tool is held at the corresponding angle. Various blade angles provide a tool that can be used at an angle ergonomically suited to the individual surgeon.

These multi-bladed scalpels are inexpensive to manufacture. They require no learning curve to use, or technician to operate. They give the surgeon more time in the time-critical part of the surgery. They decrease hand fatigue, so all incisions can be made more carefully and precisely. The parallel blades provide very repeatable incision spacing and alignment—one incision does not occur too close to another, so capillary revascularization around each follicle is maximized, and post-operative swelling and bleeding are minimized.

### DESCRIPTION OF DRAWINGS

Figures one (cross section, front view) and two (cross section, side view) show one embodiment—a three-bladed scalpel. The blades, B, are permanently mounted, parallel to each other, by the use of an attachment means to blade holder A. Blade holder A is attached to handle F by means of a setscrew H in this embodiment.

The blades are mounted at some angle θ with respect to the top of the handle. This provides for a natural grip when the surgeon places the blades in position to make incisions. An attachment means, in this embodiment thumbscrew C, is used to secure the depth gauge, D, to the handle. Thumbscrew C is inserted into the tapped hole in A by passing it through slot G.

When the adjustment means, in this embodiment an ergonomically-shaped handle grip E with internal threads, is rotated around external threads on handle A, depth gauge D is adjusted vertically until the desired length of blade extends past the tip of the depth gauge D. Thumbscrew C is then tightened to set depth gauge D.

In this embodiment, depth gauge D is tapered at the tip, and channels are cut across the tip, to provide a clear view of the blade location while still providing depth control. Also in this embodiment, the tip angle of depth gauge D is machined such the cut depth is equal for each blade.

### DETAILED DESCRIPTION OF INVENTION

There was nothing like this, so I started from scratch. There were no inventions that allowed for blade mounting close enough such that one area of scalp could be incised for hair transplantation purposes, then the tool moved to an adjacent area to repeat the process. In this embodiment, the blade holder is machined from Delrin, and all the other parts from aluminum, although newer versions might use different materials.

The blade holder is machined from medical grade Delrin, using 1/4″ diameter, 1″ long sections. These sections are machined to provide an orientation flat along the top 0.1″ of a vertically oriented section. Blade holes are then drilled into the top such that the angle of the plane defined by the blade tips is ergonomically desirable angle—in this case, 45 degrees. The blade holes get deeper as they are drilled from left to right to achieve this angle. The orientation flat is parallel with this left—right line. 4×40 holes are then tapped halfway along the length of the rod, perpendicular to the plane of the orientation flat, and on the circular face opposite the blade holes. A stainless steel setscrew is attached to the bottom of the blade holder before the holder is populated with blades.

The handle in this embodiment is 1/4″ diameter aluminum rod, 6″ long, with a 4×40 hole tapped in one end face, and the outside diameter threaded with ¼×20 threads for a distance of 1″ down from the end face with the tapped hole.

The adjustment nut is aluminum machined to a conical shape, with ridges along the length of the cone to provide for a non-slip surface. The cone angle is designed to provide an ergonomically correct surface to hold the scalpel. The inside of the adjustment nut is tapped with ¼×20 threads along the narrower half of the cone, with the remaining length of the inside diameter machined to 0.260″. In the assembly process, the cone is threaded down onto the handle, with the larger diameter end pointing towards the top of the handle where the blade holder will be attached, before the blade holder is attached.

The depth gauge is machined from aluminum, with an internal diameter of 0.260″. The top of the gauge is machined at an angle that corresponds to the angle of the plane of the blade tips. The top is tapered to minimize wall thickness, and grooved on four sides—left, right, front and back, so blade locations can easily be seen from all sides, leaving four stubs to provide depth control. A slot is machined in the side of the depth gauge, such that the slot is over the hole in the side of the Delrin section when the depth gauge is mounted so the angle of the gauge corresponds to the angle of the blades.

The gauge slides over the blade holder, a thumbscrew is inserted through the slot in the side of the gauge into the 4×40 hole in the side of the Delrin section, and made snug, but not tight. The adjustment nut is advanced or retracted until the desired amount of blade is exposed. If the gauge needs to be retracted in this process, the user simply slides the gauge back down until it hits the top side of the adjustment nut. Once the precise amount of blade extension is set, the thumbscrew is tightened, and the assembly is ready for sterilization and use.

All aluminum parts are anodized before use. Medical grade epoxy is used to mount the blades, which are attached under a 2× to 3× magnification view to make certain the blades are parallel to each other, and the orientation flat

What I claim as my invention is a multi-bladed scalpel for making incisions in skin, such that:

1. There are many blades in one scalpel, and those blade tips define a plane that is at an ergonomically desirable angle to the top plane of the handle,

2. The spacing between blades is varied by a few thousandths of an inch to minimize any appearance of a pattern.

3. It has an adjustable depth gauge, with blade placement clearly seen from all angles.

4. Blades, and therefore incisions, can be spaced as close as 0.030″ to mimic natural hair density.
